(19) United States
(12) Patent Application Publication
(10) Pub. No.: US 2024/0123192 A1
(43) Pub. Date: Apr. 18, 2024

(54) WIRE LOCK FOR MEDICAL DEVICES

(71) Applicant: St. Jude Medical, Cardiology Division, Inc., St. Paul, MN (US)

(72) Inventors: Scott Hurson, Huntington Beach, CA (US); Fermin Lupotti, Lake Forest, CA (US)

(21) Appl. No.: 18/474,732

(22) Filed: Sep. 26, 2023

Related U.S. Application Data
(60) Provisional application No. 63/417,068, filed on Oct. 18, 2022.

(51) Int. Cl. A61M 25/01

(52) U.S. Cl. CPC A61M 25/0147; A61M 2025/015

(57) ABSTRACT

A pull wire assembly for a steerable medical device can include wire lock and a wire. The wire lock includes a spool body having first and ends and first and second rims respectively around the first and second ends. A through-hole extends diametrically through the spool body. The wire enters through a distal end of the through-hole, emerges through a proximal end of the through-hole, wraps around an exterior of the spool body, re-enters into the distal end of the through-hole, and re-emerges through the proximal end of the through-hole. The wire may make multiple such passes. The wire may also be tensioned to at least partially embed into the surface of the spool body.

### CROSS-REFERENCE TO RELATED APPLICATIONS

This application claims the benefit of U.S. provisional application No. 63/417,068, filed 18 Oct. 2022, which is hereby incorporated by reference as though fully set forth herein.

### BACKGROUND

The instant disclosure relates to medical devices, such as catheters and other elongate medical devices. In particular, the instant disclosure relates to a wire lock, as may be employed to secure pull wires in steerable medical devices, such as steerable electrophysiology and intracardiac echocardiography (ICE) catheters.

Catheters are used for an ever-growing number of procedures, such as diagnostic, therapeutic, and ablative procedures, to name just a few examples. Typically, the catheter is manipulated through the patient's vasculature and to the intended site, for example, a site within the patient's heart.

Those of ordinary skill in the art will be familiar with steerable medical devices. In such catheters, the orientation and/or configuration of the distal end of the catheter can be controlled by one or more actuators, which are typically located on the catheter's control handle. Although various configurations are known, deflection at the distal end of the medical device is often achieved through the use of a pull wire secured to the deflection mechanism at one end and to the distal end of the catheter (e.g., via a pull ring embedded in the wall of the medical device) at its other end. Manipulation of the actuator places the pull wire in tension, which, in turn, effects changes in the orientation and/or configuration of the distal end of the catheter.

In many extant steerable medical devices, the proximal ends of the pull wires are secured to the deflection mechanism via the use of wire locks. For example, the proximal ends of the pull wires may be soldered to disc-shaped copper wire locks, though the use of solder connections increases the complexity of the manufacturing process.

### BRIEF SUMMARY

Aspects of the instant disclosure relate to wire lock structures that can be used, for instance, to secure pull wires to deflection mechanisms in deflectable catheters and analogous elongate medical devices. In this regard, disclosed herein is a pull wire assembly for a steerable medical device, including a wire lock and a wire. The wire lock includes: a spool body having a first end and a second end; a first rim around the first end of the spool body; a second rim around the second end of the spool body; and a through-hole extending diametrically through the spool body. The wire has an end portion that enters through a first (e.g., distal) end of the through-hole, emerges through a second (e.g., proximal) end of the through-hole opposite the first end of the through-hole, wraps around an exterior surface of the spool body, re-enters into the first end of the through-hole, and re-emerges through the second end of the through-hole. In aspects of the disclosure, after re-emerging through the second end of the through-hole, the end of the wire re-wraps around the exterior surface of the spool body, re-enters into the first end of the through hole, and re-emerges through the second end of the through-hole.

In embodiments of the disclosure, the wire is under sufficient tension to at least partially embed the wire into the exterior surface of the spool body.

It is contemplated that the spool body can be a cylindrical spool body. The first rim and second rim can be circular rims or non-circular rims. Where the first rim and/or the second rim are non-circular rims, an orientation chamber configured to receive the wire lock with the spool body in a preset orientation and to retain the spool body in the preset orientation can be utilized.

The through-hole can taper from a maximum diameter at the exterior surface of the spool body to a minimum diameter within the spool body.

Also disclosed herein is a wire lock including: a spool body having a first end and a second end; a first rim around the first end of the spool body; a second rim around the second end of the spool body; and a through-hole extending diametrically through the spool body.

It is contemplated that the spool body can be a cylindrical spool body. The first rim and the second rim can be circular rims or non-circular rims. Where the first rim and/or the second rim are non-circular rims, an orientation chamber configured to receive the spool body in a preset orientation and to retain the spool body in the preset orientation can be utilized.

The through-hole can taper from a maximum diameter at an exterior surface of the spool body to a minimum diameter within the spool body.

The instant disclosure also provides a method of manufacturing a steerable medical device. The method includes: securing a distal end of a pull wire to a body of a medical device; routing a proximal end of the pull wire through the body of the medical device to a steering assembly located at a proximal end of the medical device, wherein the steering assembly comprises a wire lock; and securing the proximal end of the pull wire to the wire lock. The wire lock includes: a spool body having a first end and a second end; a first rim around the first end of the spool body; a second rim around the second end of the spool body; and a through-hole extending diametrically through the spool body. The step of securing the proximal end of the pull wire to the wire lock includes: routing the proximal end of the pull wire into a distal end of the through-hole and out of a proximal end of the through-hole; wrapping the proximal end of the pull wire around an exterior surface of the spool body; and, after wrapping the proximal end of the pull wire around the exterior surface of the spool body, routing the proximal end of the pull wire into the distal end of the through-hole and out of the proximal end of the through hole.

The step of securing the proximal end of the pull wire to the wire lock can further include placing the pull wire into sufficient tension that the proximal end of the pull wire at least partially embeds the pull wire into the exterior surface of the spool body.

The spool body can be a cylindrical spool body, and the first rim and the second rim can be, respectively, a first circular rim and a second circular rim. Alternatively, the spool body can be a cylindrical spool body, and the first rim and the second rim can be, respectively a first non-circular rim and a second non-circular rim. Further, it is contemplated that the steering assembly may further include an orientation chamber configured to receive the spool body in a preset orientation and to retain the spool body in the preset orientation, and that securing the proximal end of the pull wire to the wire lock includes positioning the wire lock in the orientation chamber with the spool body in the preset orientation.

The through-hole can taper from a maximum diameter at the exterior surface of the spool body to a minimum diameter within the spool body.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 depicts an exemplary deflectable catheter.

FIG. 2 depicts the handle of the catheter of FIG. 1 with the exterior housing removed in order to visualize certain internal components thereof.

FIG. 3 is a close up of region 3 in FIG. 2.

FIG. 4 illustrates a wire lock according to certain embodiments of the instant disclosure.

FIG. 5 illustrates a wire lock according to additional embodiments of the instant disclosure, including the use of an orientation chamber.

FIG. 6 schematically depicts how a pull wire may be secured to a wire lock according to certain aspects of the teachings herein.

FIG. 7 depicts how a pull wire may at least partially embed into a wire lock as described in connection with aspects of the present disclosure.

### DETAILED DESCRIPTION

The instant disclosure relates to wire locks that can be used, for example, to secure pull wires within the deflection mechanism of a deflectable elongate medical device. For the sake of illustration, certain embodiments of the disclosure will be explained herein with reference to a steerable catheter, such as the ViewFlex™ Xtra Intracardiac Echocardiography Catheter (Abbott Laboratories; Abbott Park, Illinois). It should be understood, however, that the present teachings may be applied to good advantage in other contexts as well.

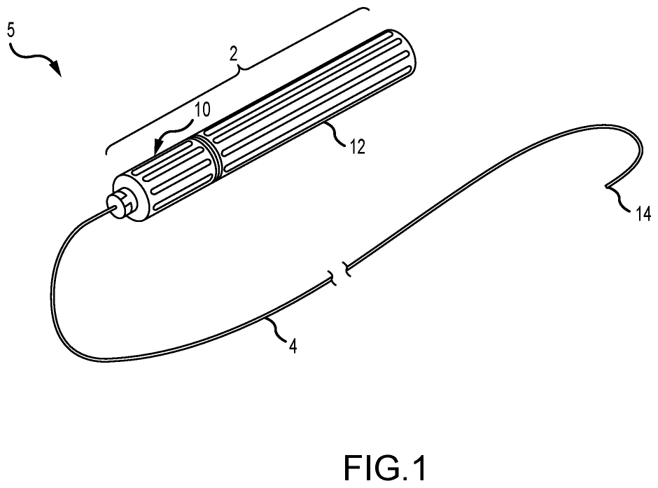

FIG. 1 depicts an exemplary steerable catheter 5. Catheter 5 generally includes a flexible tubular body 4 and a handle 2. It should be understood that the overall length of body 4 as depicted in FIG. 1 is merely exemplary and may vary consistent with the teachings herein, provided, of course, that it is sufficient to reach the intended destination within the patient's body (e.g., the heart).

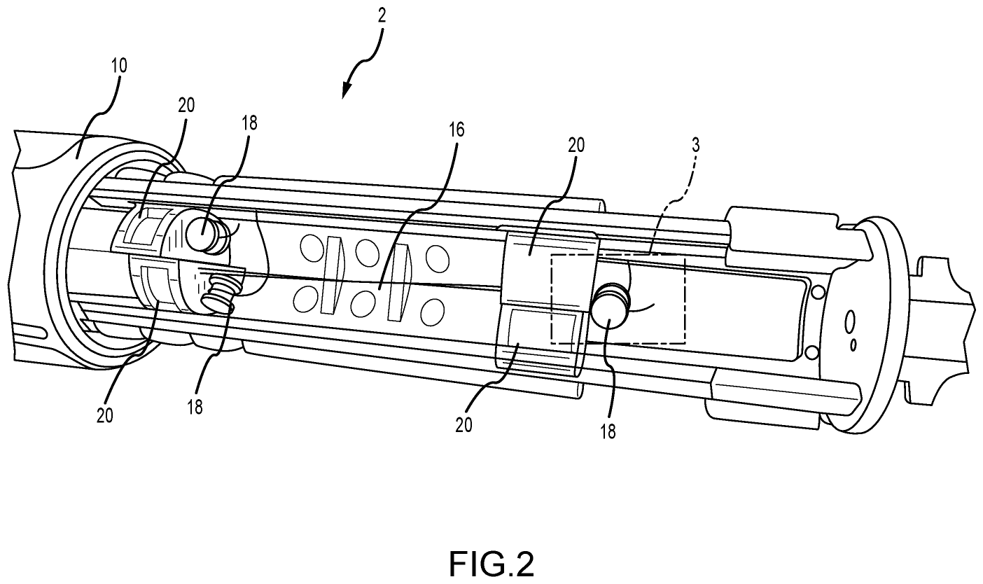

As shown in FIG. 1, handle 2 is connected to body 4 and includes an actuator 10 and a grip portion 12. As those of ordinary skill in the art will appreciate, manipulation of actuator 10 (e.g., pivoting actuator 10 in one direction or the other about the longitudinal axis of handle 2) causes distal end 14 of body 4 to deflect (e.g., bi-directionally) through the action of one or more pull wires (visible, for example, in FIG. 2) extending from within handle 2 to a point within or near distal end 14 of body 4, where they may be secured to body 4 via a pull ring or other suitable structure.

Insofar as the basic construction of steerable catheters will be familiar to those of ordinary skill in the art, it need not be, and will not be, discussed in detail herein, except as necessary to understand the instant disclosure. By way of illustration only, however, U.S. Pat. No. 8,676,290, which is hereby incorporated by reference as though fully set forth herein, describes various embodiments of catheter 5, and more particularly of handle 2, in connection with which the instant teachings may be applied to good advantage.

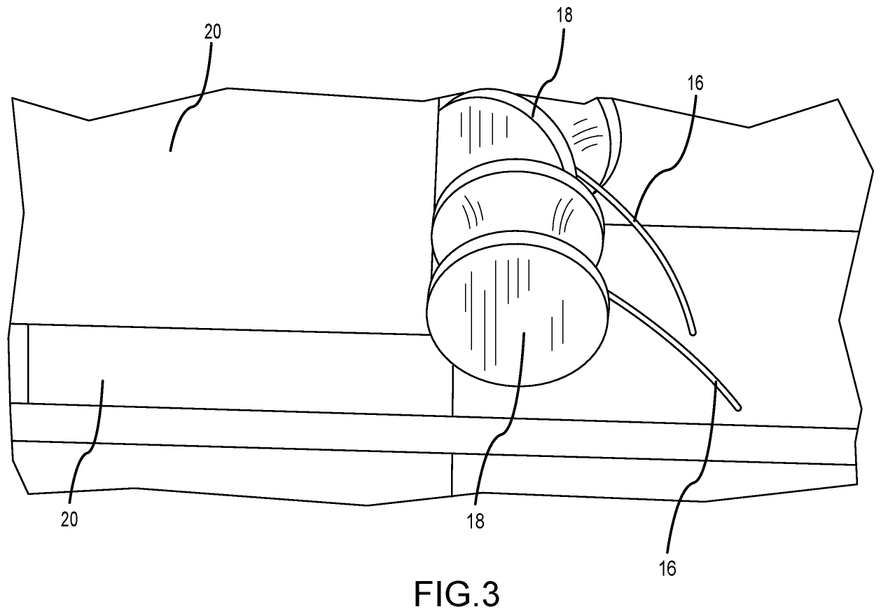

In FIG. 2, grip portion 12 of handle 2 has been removed, thus making visible a plurality of pull wires 16 within handle 2. As described in greater detail below, each pull wire 16 is secured to a wire lock 18, which in turn can ride on a respective slider block 20 of a pair of slider blocks 20 (shown in close up in FIG. 3).

Thus, as those of ordinary skill in the art will recognize, pivoting actuator 10 in one direction or the other will cause the two slider blocks 20 within a pair of slider blocks 20 to move in opposing directions (that is, one slider block 20 of the pair will move proximally, while the other slider block 20 of the pair will move distally), such as by action of a rack-and-pinion gear system within handle 2. Slider block 20 moving proximally will push wire lock 18 riding thereon proximally, which places corresponding pull wire 16 secured thereto into tension, which, in turn, deflects distal end 14 of body 4.

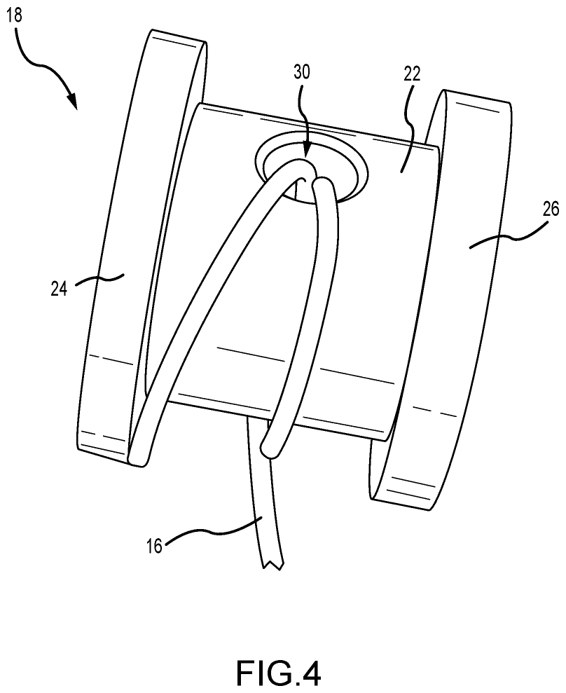

FIG. 4 illustrates a pull wire assembly (that is, the connection between pull wire 16 and wire lock 18) according to aspects of the instant disclosure. As shown in FIG. 4, wire lock 18 generally includes a spool body 22, with a first rim 24 around a first end of spool body 22 and a second rim 26 around a second end of spool body 22. First and second rims 22, 24 provide spacing between spool body 22 and slider block 20 upon which it rides.

In embodiments of the disclosure, spool body 22 is a cylindrical body. As used herein, a “cylindrical body” is not limited to a body having a circular and/or oval cross sections, as other curvatures are contemplated. Likewise, the term “cylindrical body” is not limited to right cylinders, thus allowing the opposing ends of spool body 22 to have differing planar areas. In other words, spool body 22 can taper from one end to the other, or from both ends to the center (thus creating an hourglass shape), and still be a “cylindrical body” within the meaning of the instant disclosure.

Similarly, according to aspects of the disclosure, first rim 24 and second rim 26 can both be circular rims. The use of circular rims is advantageous, insofar as it helps ensure that wire lock 18 will always ride substantially level on, and with substantially constant spacing from, slider block 20.

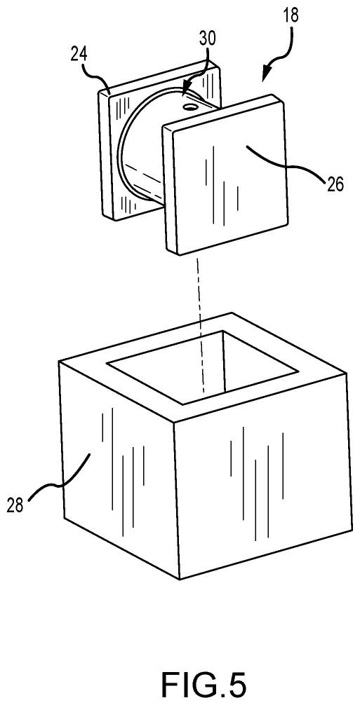

Other, non-circular shapes for first rim 24 and second rim 26, however, are also contemplated. For instance, FIG. 5 depicts an embodiment of wire lock 18 where first rim 24 and second rim 26 are square rims. Because square rims will not necessarily ride level on slider block 20, nor necessarily maintain constant spacing from slider block 20, an orientation chamber 28 can be included (e.g., affixed to slider block 20). Orientation chamber 28 is configured to receive wire lock 18 with spool body 22 in a preset orientation, and to retain spool body 22 in that preset orientation as it rides on slider block 20 (e.g., with the sides of square first and second rims 24, 26 riding level on slider block 20).

A through-hole 30 extends diametrically through spool body 22. As used herein, the phrase “extends diametrically through” (and its variants, such as “extending diametrically through”) means that through-hole 30 extends along a generally straight line across spool body 22, typically, but not necessarily, through the center of spool body 22. Thus, for example, through-hole 30 can extend along any chord of spool body 22, including the diameter of spool body 22, and be “extending diametrically through” spool body 22 within the meaning of the instant disclosure.

According to aspects of the disclosure, through-hole 30 has a generally constant diameter (or other cross-sectional dimension, insofar as through-hole 30 need not have a circular cross-section) along its entire length. In other aspects of the disclosure, however, through-hole 30 has a varying diameter (or other cross-sectional dimension) along its length. For example, through-hole 30 can have an hourglass-like shape, tapering from a maximum diameter (or other cross-sectional dimension) at the exterior surface of spool body 22 to a minimum diameter (or other cross-sectional dimension) within spool body 22. The use of a tapering through-hole 30 can facilitate passage of pull wire 16 therethrough during assembly. It may also simplify manufacture of wire lock 18, particularly where wire lock 18 is formed through a molding process where the taper will aid in releasing wire lock 18 from the mold in which it is formed.

Both metals and plastics can be used to form wire lock 18. Specifically suitable materials include, without limitation, liquid crystal polymer, brass, copper, and combinations thereof. Those of ordinary skill in the art will appreciate how to select a material, or a combination of materials, appropriate for any given application for wire lock 18.

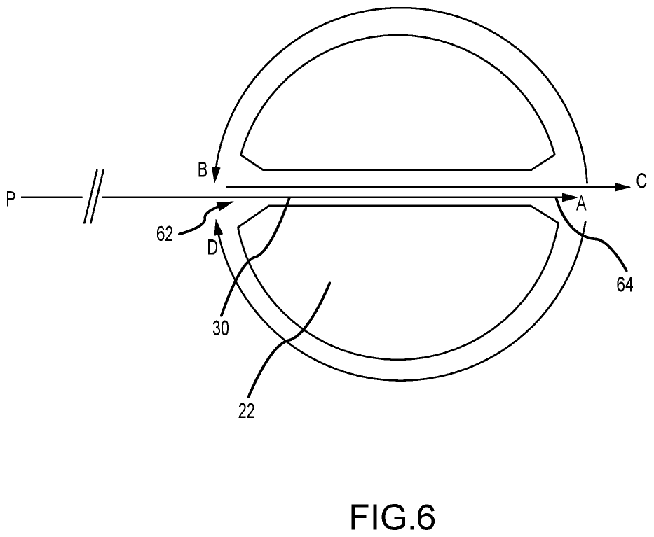

FIG. 6 schematically illustrates how pull wire 14 is secured to wire lock 18 in accordance with certain embodiments disclosed herein. First, following path A, which extends from a point P where pull wire 16 is secured to catheter body 4 (e.g., a pull ring embedded within catheter body) within or near distal end 14 and along catheter body 4 into handle 2, pull wire 16 enters the distal opening 62 of through-hole 30 and emerges from the proximal opening 64 of through-hole 30. Next, pull wire 16 wraps around the exterior surface of spool body 12 along path B, re-enters distal opening 62 of through-hole 30, and, following path C, re-emerges from proximal opening 64 of through-hole 30.

The foregoing process of wrapping pull wire 16 around the exterior surface of spool body 22 and passing it back through through-hole 30 can be repeated as many times as desired; in some embodiments of the disclosure, pull wire 16 is twice looped around the exterior surface of spool body 22. It should also be understood that, on one or more passes, pull wire 16 can be wrapped around the opposite side of the exterior surface of spool body 22 (that is, along path D).

Other wrapping patterns are also contemplated. For example, rather than wrapping halfway around spool body 22 before re-entering distal opening 62 of through-hole 30, pull wire 16 can wrap one or more times around spool body 22 before re-entering distal opening 62 of through-hole 30. Similarly, in embodiments of the disclosure, it is contemplated that, in at least some passes, pull wire 16 may re-enter proximal opening 64 of through-hole 30.

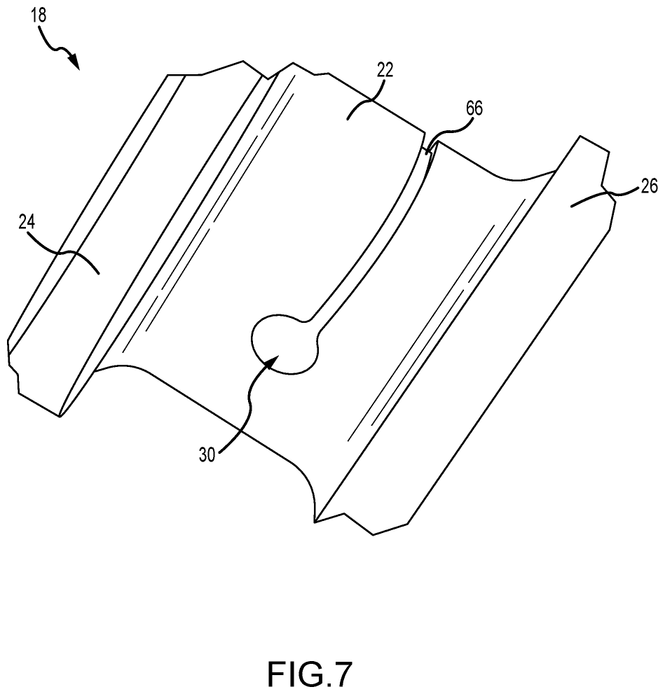

In general, however, after one or more passes as described above, pull wire 16 will be secured to wire lock 18. This security can be enhanced by pre-tensioning pull wire 16 to a point sufficient that it at least partially embeds into the exterior surface of spool body 22, thus providing additional resistance to pull wire 16 becoming disengaged from wire lock 18 when placed in tension through actuation of actuator 10. To illustrate, FIG. 7 depicts a groove 66 into the exterior surface of spool body 22 formed when pull wire 16 embeds therein (pull wire 16 is omitted from FIG. 7 in order to make groove 66 visible).

Although several embodiments of this invention have been described above with a certain degree of particularity, those skilled in the art could make numerous alterations to the disclosed embodiments without departing from the spirit or scope of this invention.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A pull wire assembly for a steerable medical device, comprising:
a wire lock comprising:
a spool body having a first end and a second end;
a first rim around the first end of the spool body;
a second rim around the second end of the spool body; and
a through-hole extending diametrically through the spool body; and
a wire having an end portion that enters through a first [distal] end of the through-hole, emerges through a second [proximal] end of the through-hole opposite the first end of the through-hole, wraps around an exterior surface of the spool body, re-enters into the first end of the through-hole, and re-emerges through the second end of the through-hole.

2. The pull wire assembly according to claim 1, wherein the wire is under tension, and wherein the tension is sufficient to at least partially embed the wire into the exterior surface of the spool body.

3. The pull wire assembly according to claim 1, wherein the spool body comprises a cylindrical spool body.

4. The pull wire assembly according to claim 3, wherein the first rim comprises a first circular rim and the second rim comprises a second circular rim.

5. The pull wire assembly according to claim 3, wherein the first rim comprises a first non-circular rim and the second rim comprises a second non-circular rim.

6. The pull wire assembly according to claim 5, further comprising an orientation chamber configured to receive the wire lock with the spool body in a preset orientation and to retain the spool body in the preset orientation.

7. The pull wire assembly according to claim 1, wherein the through-hole tapers from a maximum diameter at the exterior surface of the spool body to a minimum diameter within the spool body.

8. The pull wire assembly according to claim 1, wherein the end of the wire, after re-emerging through the second end of the through-hole, re-wraps around the exterior surface of the spool body, re-enters into the first end of the through hole, and re-emerges through the second end of the through-hole.

9. A wire lock, comprising:
a spool body having a first end and a second end;
a first rim around the first end of the spool body;
a second rim around the second end of the spool body; and
a through-hole extending diametrically through the spool body.

10. The wire lock according to claim 9, wherein the spool body comprises a cylindrical spool body.

11. The wire lock according to claim 10, wherein the first rim comprises a first circular rim and the second rim comprises a second circular rim.

12. The wire lock according to claim 10, wherein the first rim comprises a first non-circular rim and the second rim comprises a second non-circular rim

13. The wire lock according to claim 12, further comprising an orientation chamber configured to receive the spool body in a preset orientation and to retain the spool body in the preset orientation.

14. The wire lock according to claim 9, wherein the through-hole tapers from a maximum diameter at an exterior surface of the spool body to a minimum diameter within the spool body.

15. A method of manufacturing a steerable medical device, comprising:
securing a distal end of a pull wire to a body of a medical device;
routing a proximal end of the pull wire through the body of the medical device to a steering assembly located at a proximal end of the medical device, wherein the steering assembly comprises a wire lock; and
securing the proximal end of the pull wire to the wire lock,
wherein the wire lock comprises:
a spool body having a first end and a second end;
a first rim around the first end of the spool body;
a second rim around the second end of the spool body; and
a through-hole extending diametrically through the spool body; and
wherein securing the proximal end of the pull wire to the wire lock comprises:
routing the proximal end of the pull wire into a distal end of the through-hole and out of a proximal end of the through-hole;
wrapping the proximal end of the pull wire around an exterior surface of the spool body; and, after wrapping the proximal end of the pull wire around the exterior surface of the spool body,
routing the proximal end of the pull wire into the distal end of the through-hole and out of the proximal end of the through hole.

16. The method according to claim 15, wherein securing the proximal end of the pull wire to the wire lock further comprises placing the pull wire into sufficient tension that the proximal end of the pull wire at least partially embeds the pull wire into the exterior surface of the spool body.

17. The method according to claim 15, wherein the spool body comprises a cylindrical spool body, the first rim comprises a first circular rim, and the second rim comprises a second circular rim.

18. The method according to claim 15, wherein the spool body comprises a cylindrical spool body, the first rim comprises a first non-circular rim, and the second rim comprises a second non-circular rim.

19. The method according to claim 18, wherein the steering assembly further comprises an orientation chamber configured to receive the spool body in a preset orientation and to retain the spool body in the preset orientation, and wherein securing the proximal end of the pull wire to the wire lock further comprises positioning the wire lock in the orientation chamber with the spool body in the preset orientation.

20. The method according to claim 15, wherein the through-hole tapers from a maximum diameter at the exterior surface of the spool body to a minimum diameter within the spool body.
